(19) United States
(12) Patent Application Publication
(10) Pub. No.: US 2008/0153917 A1
(43) Pub. Date: Jun. 26, 2008

(54) Sigma Receptor Ligands

(75) Inventors: James Ellis, Boxford, MA (US); Doina Ene, Lisbon, CT (US); Eric Schwartz, Wakefield, MA (US); Nivedita Namdev, Westford, MA (US); Claire Brown, Medford, MA (US); Sophie Binet-Cross, Lynn, MA (US); David Meyers, Towson, MD (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 11/574,604

(22) PCT Filed: Sep. 9, 2005
(86) PCT No.: PCT/EP05/09682

§ 371 Date: Jul. 10, 2007

Related U.S. Application Data
(63) Continuation of application No. 10/939,149, filed on Sep. 10, 2004, now abandoned.

(51) Int. Cl. A61K 31/165; C07C 233/34; A61P 11/14; A61P 25/28; A61P 25/24; A61P 37/00; A61P 29/00

(52) U.S. Cl. 514/617; 564/161

(57) ABSTRACT

The present invention concerns phenylcyclopentylacetamides and phenylcyclopropylcarboxamides and analogues thereof, processes for preparing them, pharmaceutical compositions containing them and their use as pharmaceuticals.

The present invention concerns phenylcyclopentylacetamides and phenylcyclopropylcarboxamides and analogues thereof, processes for preparing them, pharmaceutical compositions containing them and their use as pharmaceuticals.

The Sigma receptor is a ubiquitous transmembrane receptor whose function has not been well defined but which is highly conserved across mammalian species/tissues. The invention relates to novel sigma receptor ligands of either sigma 1 or sigma 2 receptor subtype and these compounds have uses as either agonists or antagonists. As such they will have utility in modifying states where sigma receptors are thought to be involved. Owing to the high concentration of sigma receptors in CNS tissue, many reported functions of the sigma receptor involve neuromodulation, however, additional peripheral actions of sigma have also been reported, suggesting a widespread function of these receptors. (DeHaven-Hudkins et al. Biochem Pharmacol 1994, 47:1231-1239; Pascaud et al. J Pharmacol Exp Ther. 1990 Dec; 255(3):1354-9; Liu, et al., J Neuroimmunol. 1995 June; 59(1-2):143-54).

Conventional treatment for cough has been achieved using codeine or dextromethorphan which are believed to inhibit cough via a central mechanism of action. This invention targets the use of specific sigma receptor ligands as novel antitussives. Since there is evidence for a role for sigma receptors in various CNS functions (Su et al., Critical reviews in Neurobiology 1993, 7 (3/4): 187-203) other potential applications of sigma receptor ligands include the treatment of CNS disease states and disorders such as Alzheimer's disease, depression, anxiety, psychosis, stress, senescence and memory impairment as well as in immune modulation and inflammation (Liu, et al., J Neuroimmunol. 1995 Jun;59(1-2):143-54) and treatment of GI disorders such as diarrhea.

The present invention therefore provides novel phenylcyclopentylacetamides and phenylcyclopropylcarboxamides as compounds that are useful as sigma receptor ligands (agonists and antagonists) in the treatment of cough or in the treatment of diseases of cognitive dysfunction. The compounds disclosed herein can also be used as research tools to study biological pathways involving the sigma-1 receptor.

Compounds N-3-[-(dimethylamino)propyl]-2-phenyl-cyclopropanecarboxamide; N-[2-(4-morpholinyl)ethyl]-2-phenyl-cyclopropanecarboxamide; N-2-[-(dimethylamino) ethyl]-2-phenyl-cyclopropanecarboxamide; N-[3-(4-morpholinyl)propyl]-2-phenyl-cyclopropane carboxamide; 2-phenyl-N-[2-(1-piperidinyl)ethyl]-cyclopropanecarboxamide are disclosed in chemical libraries.

N-(4-aminobutyl)-2-phenyl-cyclopropanecarboxamidisclosed in patent application WO9931064, which describes useful cytoststic and immunosuppressive agents.

1-[[(1R,2R)-2-phenyl cyclopropyl] carbonyl]-4-piperidinemethanamine mono-trifluoroacetate is disclosed in patent application WO 02/068409 as NMDA/NR2B antagonists useful for relieving pain.

2-Cyclopentyl-N-[3-(2,6-dimethyl-piperidin-1-yl)-propyl]-2-phenyl-acetamide is described in Journal of Med. Chem. 1994, 37 (2), 268-274 as a sodium channel blocker.

2-Cyclopentyl-N-(1-diethylaminomethyl-cyclohexyl)-2-phenylacetamide is described in Scienta Pharmaceutica 1999, 58(3), 273-280 for its antiemetic activity.

European patent application EP 0383256 discloses substituted-acetamide compounds and a process for their preparation.

International patent application WO96/40136, Bioorg. Med. Chem. Lett. 10 (2000) 1621 -1624 and U.S. Pat. No. 5,977,115 disclose phenylcyclopentylacetamide derivatives as alpha-1a adrenergic receptor antagonists.

Japanese patent application JP2002047272 discloses polyamine amide derivatives for certain CNS disorders.

Journal de Pharmacie de Belgique 1997, 52(2), 57-60 describes phenylcyclopentylcarboxamide derivatives as anticonvulsants.

International patent application WO2001077101 discloses phenylcyclopentylacetamide derivatives for the treatment of a chemokine or H1 related disease.

In one aspect, the invention provides a compound having formula I or a pharmaceutically acceptable salt thereof or stereoisomeric forms thereof, and the geometrical isomers, enantiomers, diastereoisomers, and pharmaceutically acceptable salts thereof or their corresponding N-oxides

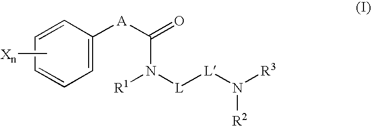

wherein,

X is halogen;

n is 0 to 5;

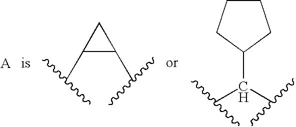

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

except compounds N-3-[-(dimethylamino) propyl]-2-phenyl-cyclopropane carboxamide; N-[2-(4-morpholinyl)ethyl]-2-phenyl-cyclopropanecarboxamide; N-2-[- (dimethyl amino) ethyl]-2-phenyl-cyclopropanecarboxamide; N-[3-(4-morpholinyl) propyl]-2-phenyl-cyclopropane carboxamide; 2-phenyl-N-[2-(1-piperidinyl)ethyl]- cyclopropanecarboxamide; (1R,2R)-N-(4-aminobutyl)-2-phenyl-cyclopropanecarboxamide; 1-[[(1R,2R)-2-phenyl cyclopropyl] carbonyl]-4-piperidine methanamine mono (trifluoroacetate); N-(4-aminobutyl)-2-phenyl-cyclopropanecarboxamide; 2-cyclopentyl-N-[3-(2,6-dimethyl-piperidin-1-yl)-propyl]-2-phenyl-acetamide; 2-cyclopentyl-N-(1-diethylaminomethyl-cyclohexyl)-2-phenyl-acetamide.

In another aspect the invention provides a compound having formula II or a pharmaceutically acceptable salt thereof or stereoisomeric forms thereof, and the geometrical isomers, enantiomers, diastereoisomers, and pharmaceutically acceptable salts thereof or their corresponding N-oxides

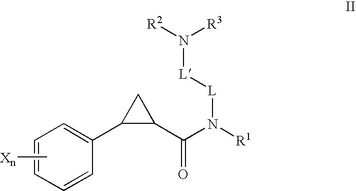

wherein,

X is halogen;

n is 0 to 5;

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

except N-3-[-(dimethylamino) propyl]-2-phenyl-cyclopropanecarboxamide; N-[2-(4-morpholinyl)ethyl]-2-phenyl-cyclopropanecarboxamide; N-2-[-(dimethyl amino)ethyl]-2-phenyl-cyclopropanecarboxamide; N-[3-(4-morpholinyl) propyl]-2-phenyl-cyclopropane carboxamide; 2-phenyl-N-[2-(1-piperidinyl)ethyl]-cyclopropanecarboxamide; (1R,2R)-N-(4-aminobutyl)-2-phenyl-cyclopropanecarboxamide; 1-[[(1R,2R)-2-phenylcyclopropyl] carbonyl]-4-piperidine methanamine mono (trifluoroacetate); N-(4-aminobutyl)-2-phenyl-cyclopropanecarboxamide.

In another aspect the invention provides a compound having formula III or a pharmaceutically acceptable salt thereof or stereoisomeric forms thereof, and the geometrical isomers, enantiomers, diastereoisomers, and pharmaceutically acceptable salts thereof or their corresponding N-oxides

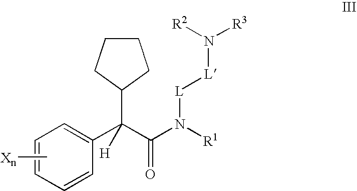

wherein,

X is halogen;

n is 0 to 5;

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

except 2-cyclopentyl-N-[3-(2,6-dimethyl-piperidin-1-yl)-propyl]-2-phenylacetamide; 2-2yclopentyl-N-(1-diethylaminomethyl-cyclohexyl)-2-phenylacetamide.

The term “alkyl”, as used herein, is defined as including a saturated, monovalent hydrocarbon moiety having straight or branched moieties or combinations thereof and containing 1-8 carbon atoms, preferably 1-6 carbon atoms and more preferably 1-4 carbon atoms. Alkyl moieties can optionally be substituted by cycloalkyl groups or one methylene can be replaced by a carbonyl or carboxyl group. Usually, in the present case, alkyl groups are methyl, ethyl, isopropyl, ethylacetyl, methylacetate. Preferred alkyl groups are methyl, ethyl, isopropyl. Most preferred alkyl group is ethyl.

The term “alkylene”, as used herein, is defined as including saturated, divalent hydrocarbon moieties having straight or branched moieties or combinations thereof and containing 1-8 carbon atoms, preferably 1-6 carbon atoms and more preferably 1-4 carbon atoms. Usually alkylene groups are methylene, ethylene, n-propylidene, 2,2-dimethylpropylidene, n-butylidene. Preferred alkylene groups are ethylene, n-propylene.

The term “cycloalkyl”, as used herein, refers to a monovalent or divalent group of 3 to 8 carbon atoms, preferably 3-6 carbon atoms derived from a saturated cyclic hydrocarbon. Cycloalkly groups can be optionally substituted by alkyl groups. Usually a cycloalkyl groups are cyclohexyl, methylcyclopropyl, cyclopentyl, cyclopropyl. Preferred cycloalkyl groups are cyclohexyl, cyclopentyl and cyclopropyl.

The term “halogen”, as used herein, includes an atom of Cl, Br, F, I. Usually, halogens are Cl, F.

The term “heterocyclic ring”, as used herein is defined as including a non aromatic cycloalkyl moiety as defined above, having at least one O and/or N atom interrupting the carbocyclic ring structure. Heterocyclic ring moieties can optionally be substituted by aryl, hydroxyl or alkyl groups. Usually heterocyclic groups, in the present case are, piperazinyl, piperidinyl, morpholinyl, pyrrolidinyl, methyl-1-piperidinyl, 4-methylpiperazinyl, 4-(2-chloro-6-fluorobenzyl)-1-piperazinyl, 4-(3,4-dichlorobenzyl)-1-piperazinyl, 4-isopropylpiperazinyl, 4-methylacetatepiperidinyl, 4-ethylacetylpiperidinyl, 4-hydroxylpiperidinyl, 4-(2-chloro-6-fluorobenzoyl)-1-piperazyl. Preferred heterocyclic groups are piperidinyl, pyrrolidinyl, 4-(2-chloro-6-fluorobenzyl)-1-piperazinyl, 4-(3,4-dichlorobenzyl)-1-piperazinyl. Most preferred heterocyclic groups are piperidinyl and pyrrolidinyl.

The term “aryl”, as used herein, is defined as including an organic moiety derived from an aromatic hydrocarbon consisting of a ring containing 6 carbon atoms by removal of one hydrogen, such as phenyl optionally substituted by 1 to 2 halogen substituents. The aryl moiety can be directly attached to the rest of the molecule or via an alkylene (in the case of benzyl) or via a carbonyl group. Usually, in the present case, aryl groups are phenyl, benzyl, 2-chloro-6-fluorobenzyl, 3,4-dichlorobenzyl, 2-chloro-6-fluorobenzoyl, 4-fluorophenyl.

The term “hydroxyl”, as used herein, represents a group of formula —OH.

The term “carbonyl”, as used herein, represents a group of formula —C═O—.

The term “carboxyl” as used herein, represents a group of formula —C(O)O—.

The term “pharmaceutically acceptable salts” refers to salts or complexes that retain the desired biological activity of the above identified compounds and exhibit minimal or no undesired toxicological effects. Effects of such salts include, but are not limited to acid addition, salts formed with inorganic acids (for example, hydrochloric acid, hydrobromic acid, sulfuric acid, phosphoric acid, nitric acid and the like), and salts formed with organic acids such as fumaric acid, maleic acid, oxalic acid, tartric acid, succinic acid, malic acid, ascorbic acid, benzoic acid, tannic acid, pamoic acid.

Generally, R¹ is hydrogen or forms together with L, a heterocyclic ring. Usually R¹ is hydrogen or together with L forms a piperidyl or pyrrolidyl ring. Preferably R¹ is hydrogen or a piperidyl ring together with L.

Generally, R² is selected from hydrogen, C₁₋₈ alkyl, C₃₋₈ cycloalkyl or form together a heterocyclic ring. Usually R² is hydrogen, C₁₋₆ alkyl, C₃₋₆ cycloalkyl, or forms together with R³ a heterocyclic ring. Preferably R² is hydrogen, ethyl, isopropyl, cyclohexyl, methylcyclohexyl, 2-methylpiperidyl, piperidyl, pyrrolidyl, 4-(2-chloro-6-fluorobenzyl)-1-piperazyl, 4-(3,4-dichlorobenzyl)-1-piperazyl. More preferably R² is ethyl, piperidyl, pyrrolidyl, hydrogen, cyclohexyl.

Generally, R³ is selected from hydrogen, C₁₋₈ alkyl, C₃₋₈ cycloalkyl or form together a heterocyclic ring. Usually R³ is hydrogen, C₁₋₆ alkyl, C₃₋₆ cycloalkyl, or forms together with R² a heterocyclic ring. Preferably R³ is hydrogen, ethyl, isopropyl, cyclohexyl, methylcyclohexyl, 2-methylpiperidyl, piperidyl, pyrrolidyl, 4-(2-chloro-6- fluorobenzyl)-1-piperazyl, 4-(3,4-dichlorobenzyl)-1-piperazyl. More preferably R³ is ethyl, piperidyl, pyrrolidyl, hydrogen, cyclohexyl.

Generally X is halogen. Usually X is Cl, F.

Generally L is independently selected from C₁₋₈ alkylene, C₃₋₈ cycloalkyl or a direct bond or L and R¹ can form together a heterocyclic ring. Usually L is C₁₋₆ alkylene, C₃₋₆ cycloalkyl, direct bond or L and R¹ can form together a heterocyclic ring. Preferably L is n-propylene, ethylene, direct bond, methylene, piperidyl.

Generally L′ is independently selected from C₁₋₈ alkylene, C₃₋₈ cycloalkyl. Usually L′ is C₁₋₆ alkylene, C₃₋₆ cycloalkyl, or a direct bond. Preferably L′ is n-propylene, ethylene, direct bond, methylene, piperidyl. with the proviso that L and L′ are not both direct bonds.

Generally “n” is 0 to 5. Usually “n” is 0, 1 or 2.

Preferred compounds are: hydrochloride salt of 2-cyclopentyl-N-[3-(2-methyl-1-piperidinyl)propyl]-2-phenylacetamide; hydrochloride salt of 2-cyclopentyl-N-[2-(diethylamino)ethyl]-2-phenylacetamide; hydrochloride salt of (−)2-cyclopentyl-N-[2-(diethylamino)ethyl]-2-phenylacetamide; hydrochloride salt of (+)2-cyclopentyl-N-[2-(diethylamino)ethyl]-2-phenylacetamide; hydrochloride salt of trans-N-[2-(diisopropylamino)ethyl]-2-phenylcyclopropanecarboxamide; hydrochloride salt of 2-cyclopentyl-N-[2-(diethylamino)ethyl]-2-(4-fluorophenyl)acetamide; hydrochloride salt of 2-cyclopentyl-N-[3-(diethylamino)propyl]-2-phenylacetamide; hydrochloride salt of 1′-[cyclopentyl(phenyl)acetyl]-1,4′-bipiperidine; hydrochloride salt of 1′-[cyclopentyl(3,4-dichlorophenyl)acetyl]-1,4′-bipiperidine; hydrochloride salt of 1-[cyclopentyl(phenyl)acetyl]-4-pyrrolidin-1-ylpiperidine; hydrochloride salt of 1′-[(4-chlorophenyl)(cyclopentyl)acetyl]-1,4′-bipiperidine; hydrochloride salt of 2-cyclopentyl-2-phenyl-N-[2-(1-pyrrolidinyl)ethyl]acetamide; hydrochloride salt of trans-N-{3-[4-(2-chloro-6-fluorobenzyl)-1-piperazinyl]propyl}-2-cyclopentyl-2-phenylacetamide; hydrochloride salt of trans-N-[3-(cyclohexylamino)propyl]-2-phenylcyclopropanecarboxamide; hydrochloride salt of trans-1′-[(2-phenylcyclopropyl)carbonyl]-1,4′-bipiperidine; trans-2-phenyl-N-(2-pyrrolidin-1-ylethyl)cyclopropanecarboxamide; hydrochloride salt of 2-cyclopentyl-2-phenyl-N-[3-(1-pyrrolidinyl)propyl]acetamide; hydrochloride salt of trans-N-{3-[4-(2-chloro-6-fluorobenzyl)-1-piperazinyl]propyl}-2-phenylcyclopropanecarboxamide; hydrochloride salt of trans-N-[2-(4-benzyl-1-piperazinyl)ethyl]-2-phenylcyclopropanecarboxamide; hydrochloride salt of trans-N-(cyclohexylmethyl){1-[(2-phenylcyclopropyl)carbonyl]-4-piperidinyl}methanamine; hydrochloride salt of trans-N-{2-[4-(3,4-dichlorobenzyl)-1-piperazinyl]ethyl}-2-phenylcyclopropanecarboxamide; hydrochloride salt of trans-1′-[(2-phenylcyclopropyl)carbonyl]-1,4′-bipiperidine; hydrochloride salt of trans-N-[2-(diethylamino)ethyl]-2-phenylcyclopropanecarboxamide; hydrochloride salt of N-[3-(cyclohexylamino)propyl]-2-cyclopentyl-2-phenylacetamide; hydrochloride salt of trans-(+)N-[3-(cyclohexylamino)propyl]-2-phenylcyclopropanecarboxamide; hydrochloride salt of trans-(−)N-[3-(cyclohexylamino)propyl]-2-phenylcyclopropane carboxamide.

Most preferred compounds are: hydrochloride salt of 2-cyclopentyl-N-[2-(diethylamino)ethyl]-2-phenylacetamide; hydrochloride salt of (−) 2-cyclopentyl-N-[2-(diethylamino)ethyl]-2-phenylacetamide; hydrochloride salt of (+) 2-cyclopentyl-N-[2-(diethylamino)ethyl]-2-phenylacetamide; hydrochloride salt of 2-cyclopentyl-N-[3-(diethylamino)propyl]-2-phenylacetamide; hydrochloride salt of 1′-[cyclopentyl(phenyl)acetyl]-1,4′-bipiperidine; hydrochloride salt of 1′-[cyclopentyl(3,4-dichlorophenyl)acetyl]-1,4′-bipiperidine; hydrochloride salt of 1-[cyclopentyl(phenyl)acetyl]-4-pyrrolidin-1-ylpiperidine; hydrochloride salt of trans-N-[3-(cyclohexylamino)propyl]-2-phenylcyclopropanecarboxamide; hydrochloride salt of trans-1′-[(2-phenylcyclopropyl)carbonyl]-1,4′-bipiperidine; hydrochloride salt of trans-(+)N-[3-(cyclohexylamino)propyl]-2-phenylcyclopropanecarboxamide; hydrochloride salt of trans-(−)N-[3-(cyclohexylamino)propyl]-2-phenylcyclopropanecarboxamide.

TABLE OF COMPOUNDS OF FORMULA (I)

| | | | | M⁺ |
|---|---|---|---|---|
| | | Salt | Melting | Observed |
| | IUPAC Name | for M | Point (° C.) | (M⁺ + 1) |
| 1 | Trans-N-[2-(diisopropylamino)ethyl]-2- | HCl | 175 | 289 |
| | phenylcyclopropanecarboxamide | | | |
| 2 | Trans-N-[3-(cyclohexylamino)propyl]-2- | HCl | 178 | 301 |
| | phenylcyclopropanecarboxamide | | | |
| 3 | Trans-1′-[(2-phenylcyclopropyl)carbonyl]-1,4′- | HCl | 86 | 313 |
| | bipiperidine | | | |
| 4 | Trans-2-phenyl-N-(2-pyrrolidin-1- | na | 48 | 259 |
| | ylethyl)cyclopropanecarboxamide | | | |
| 5 | Trans-N-{3-[4-(2-chloro-6-fluorobenzyl)-1- | 2 HCl | 145 | 430.1 |
| | piperazinyl]propyl}-2- | | | |
| | phenylcyclopropanecarboxamide | | | |
| 6 | Trans-1-[(2-phenylcyclopropyl)carbonyl]-4-(1- | na | na | 299 |
| | pyrrolidinyl)piperidine | | | |
| 7 | Trans-N-[2-(4-benzyl-1-piperazinyl)ethyl]-2- | 2 HCl | 195 | 364 |
| | phenylcyclopropanecarboxamide | | | |
| 8 | Trans-N-(cyclohexylmethyl){1-[(2- | HCl | na | 355 |
| | phenylcyclopropyl)carbonyl]-4- | | | |
| | piperidinyl}methanamine | | | |
| 9 | Trans-N-{2-[4-(2-chloro-6-fluorobenzoyl)-1- | HCl | 122 | 430 |
| | piperazinyl]ethyl}-2- | | | |
| | phenylcyclopropanecarboxamide | | | |
| 10 | Trans-N-{2-[4-(3,4-dichlorobenzyl)-1- | 2 HCl | 145 | 432 |
| | piperazinyl]ethyl}-2- | | | |
| | phenylcyclopropanecarboxamide | | | |
| 11 | Trans-N-{2-[4-(2-chloro-6-fluorobenzyl)-1- | 2 HCl | 110 | 416 |
| | piperazinyl]ethyl}-2- | | | |
| | phenylcyclopropanecarboxamide | | | |
| 12 | Trans-1′-{[2-(4- | HCl | na | 331 |
| | fluorophenyl)cyclopropyl]carbonyl}-1,4′- | | | |
| | bipiperidine | | | |
| 13 | Trans-2-phenyl-N-[4-(1- | HCl | 129 | 327 |
| | piperidinyl)cyclohexyl]cyclopropanecarboxamide | | | |
| 14 | 2-cyclopentyl-N-[3-(2-methyl-1- | HCl | 69 | 343 |
| | piperidinyl)propyl]-2-phenylacetamide | | | |
| 15 | 2-cyclopentyl-N-[2-(diethylamino)ethyl]-2- | HCl | 129 | 303 |
| | phenylacetamide | | | |
| 16 | (−)2-cyclopentyl-N-[2-(diethylamino)ethyl]-2- | HCl | 144 | 303 |
| | phenylacetamide | | | |
| 17 | (+)2-cyclopentyl-N-[2-(diethylamino)ethyl]-2- | HCl | 146 | 303 |
| | phenylacetamide | | | |
| 18 | 2-cyclopentyl-N-[2-(ethylamino)ethyl]-2- | HCl | 131 | 275 |
| | phenylacetamide | | | |
| 19 | 2-cyclopentyl-N-[2-(diisopropylamino)ethyl]-2- | HCl | na | 331 |
| | phenylacetamide | | | |
| 20 | (2S)-1-[cyclopentyl(phenyl)acetyl]-2-(1- | HCl | na | 341 |
| | pyrrolidinylmethyl)pyrrolidine | | | |
| 21 | 2-cyclopentyl-N-[2-(diethylamino)ethyl]-2-(4- | HCl | 45 | 321 |
| | fluorophenyl)acetamide | | | |
| 22 | N-(2-aminoethyl)-2-cyclopentyl-2- | HCl | 290 | 247 |
| | phenylacetamide | | | |
| 23 | 2-cyclopentyl-N-[3-(diethylamino)propyl]-2- | HCl | na | 317 |
| | phenylacetamide | | | |
| 24 | 1′-[cyclopentyl(phenyl)acetyl]-1,4′-bipiperidine | HCl | 112 | 355 |
| 25 | 1′-[cyclopentyl(3,4-dichlorophenyl)acetyl]-1,4′- | HCl | 230 | 423 |
| | bipiperidine | | | |
| 26 | 1-[cyclopentyl(phenyl)acetyl]-4-pyrrolidin-1- | HCl | na | 341 |
| | ylpiperidine | | | |
| 27 | 1′-[(4-chlorophenyl)(cyclopentyl)acetyl]-1,4′- | HCl | 105 | 389 |
| | bipiperidine | | | |
| 28 | 2-cyclopentyl-2-phenyl-N-[2-(1- | HCl | na | 301 |
| | pyrrolidinyl)ethyl]acetamide | | | |
| 29 | 2-cyclopentyl-2-(3,4-dichlorophenyl)-N-[2- | HCl | na | 371 |
| | (diethylamino)ethyl]acetamide | | | |
| 30 | 2-cyclopentyl-2-phenyl-N-[4-(1- | HCl | na | 329 |
| | pyrrolidinyl)butyl]acetamide | | | |
| 31 | 2-cyclopentyl-N-[3-(4-methyl-1- | HCl | na | 344 |
| | piperazinyl)propyl]-2-phenylacetamide | | | |
| 32 | 2-cyclopentyl-N-[3-(4-morpholinyl)propyl]-2- | HCl | 145 | 331 |
| | phenylacetamide | | | |
| 33 | 2-cyclopentyl-N-[3-(dimethylamino)-2,2- | HCl | na | 317 |
| | dimethylpropyl]-2-phenylacetamide | | | |
| 34 | N-{3-[4-(2-chloro-6-fluorobenzyl)-1- | 2 HCl | 148 | 472.2 |
| | piperazinyl]propyl}-2-cyclopentyl-2- | | | |
| | phenylacetamide | | | |
| 35 | 2-cyclopentyl-2-phenyl-N-[3-(1- | HCl | na | 315 |
| | pyrrolidinyl)propyl]acetamide | | | |
| 36 | 4-{1-[cyclopentyl(phenyl)acetyl]-4- | na | 111.8 | 357.3 |
| | piperidinyl}morpholine | | | |
| 37 | N-{1-[cyclopentyl(phenyl)acetyl]-4-piperidinyl}- | HCl | 79 | 343.3 |
| | N,N-diethylamine | | | |
| 38 | 1-{1-[cyclopentyl(phenyl)acetyl]-4-piperidinyl}- | na | 109.6 | 398.3 |
| | 4-isopropylpiperazine | | | |
| 39 | [1′-(2-cyclopentyl-2-phenylacetyl)-1,4′- | na | na | 427.3 |
| | bipiperidin-4-yl]methyl acetate | | | |
| 40 | ethyl 1′-[cyclopentyl(phenyl)acetyl]-1,4′- | HCl | 88 | 427.3 |
| | bipiperidine-4-carboxylate | | | |
| 41 | 1′-[cyclopentyl(phenyl)acetyl]-1,4′-bipiperidin-4- | HCl | 90 | 371 |
| | ol | | | |
| 42 | (3S)-1-[cyclopentyl(phenyl)acetyl]-N,N- | na | 68 | 301 |
| | dimethyl-3-pyrrolidinamine | | | |
| 43 | 2-cyclopentyl-N-[2-(diethylnitroryl)ethyl]-2- | na | na | 319 |
| | phenylacetamide | | | |
| 44 | N-[3-(dimethylamino)-2,2-dimethylpropyl]-2- | HCl | na | 275 |
| | phenylcyclopropanecarboxamide | | | |
| 45 | N-[3-(cyclohexlyamino)propyl]-2-cyclopentyl-2- | HCl | 45 | 343 |
| | phenylacetamide | | | |
| 46 | Trans-N-[2-(diethylamino)ethyl]-2- | HCl | na | 261 |
| | phenylcyclopropanecarboxamide | | | |
| 47 | N-{1-[(diethylamino)methyl]cyclopropyl}-2- | HCl | na | na |
| | phenylcyclopropanecarboxamide | | | |
| 48 | (+)Trans-N-[3-(cyclohexylamino)propyl]-2- | HCl | 217 | 301 |
| | phenylcyclopropanecarboxamide | | | |
| 49 | (−)Trans-N-[3-(cyclohexylamino)propyl]-2- | HCl | 216 | 301 |
| | phenylcyclopropanecarboxamide | | | |
| 50 | (+)2-cyclopentyl-N-[2-(diethylamino)ethyl]-2- | HCl | 144 | 303 |
| | phenylacetamide | | | |
| 51 | (−)2-cyclopentyl-N-[2-(diethylamino)ethyl]-2- | HCl | 146 | 303 |
| | phenylacetamide | | | |

The IUPAC name was generated with ACD 7.0 version.

The melting point was determined on Fisher-Johns melting point apparatus.

The molecular ion (M⁺+1) was detected on the Platform II, single quadruple mass spectrometer (Micromass).

“na” means not available depending on the case it means that the compound is a free base or an oil.

The following synthetic schemes illustrate how compounds according to the invention can be made. Those skilled in the art will be routinely able to modify and/or adapt the following scheme to synthesize any compound of the invention covered by formula I.

Some compounds of this invention can generally be prepared in one step from commercially available or literature starting materials as shown below via amide forming reactions.

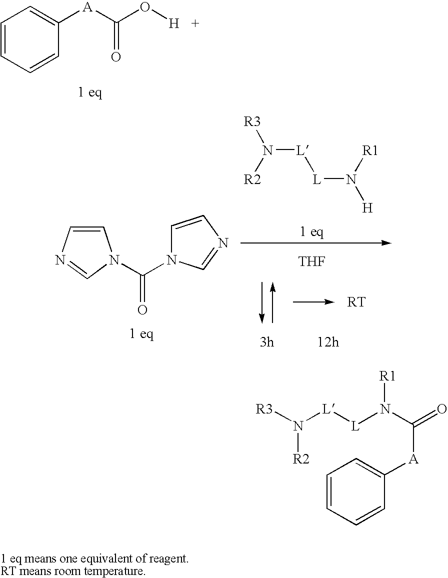

The reaction can be carried out in standard reaction conditions for amide formation (refer to “Advanced Organic Chemistry” Jerry March), starting with a carboxylic acid. The amine (usually commercially available) can be primary or secondary.

As will be evident to those skilled in the art, individual isomeric forms can be obtained by separation of mixtures thereof in conventional manner.
For example, in the case of diasteriomeric isomers, chromatographic separation may be employed.

For separation of individual optical isomers from racemic forms (for example racemic mixtures) chiral chromatography can be employed.

The compounds of the invention and compounds: N-3-[-(dimethylamino)propyl]-2-phenyl-cyclopropanecarboxamide; N-[2-(4-morpholinyl)ethyl]-2-phenyl-cyclopropanecarboxamide; N-2-[-(dimethyl amino) ethyl]-2-phenyl-cyclopropanecarboxamide; N-[3-(4-morpholinyl) propyl]-2-phenyl-cyclopropane carboxamide; 2-phenyl-N-[2-(1-piperidinyl)ethyl]-cyclopropanecarboxamide; N-(4-aminobutyl)-2-phenyl-cyclopropanecarboxamide; 1-[[(1R,2R)-2-phenyl cyclopropyl] carbonyl]-4-piperidine methanamine mono-trifluoroacetate; 2-cyclopentyl-N-[3-(2,6-dimethyl-pipridin-1-yl]-2-phenylacetamide; 2-cyclopentyl-N-(1-diethylaminomethyl-cyclohexyl)-2-phenyl-acetamide are indicated for use in treating or preventing conditions in which there is likely to be a component involving the sigma receptor ligands.

The compounds of the invention are useful as pharmaceutical preparations containing sigma 1 receptor ligands as treatment for cough. The methods of the invention comprise administration to a mammal (preferably human) suffering from cough a pharmaceutical composition according to the invention in an amount sufficient to alleviate or minimize the cough.

The compounds of the invention are useful as pharmaceutical preparations containing sigma 1 receptor ligands as treatment for diseases of cognitive dysfunction. The methods of the invention comprise administration to a mammal (preferably human) suffering from a disease of cognitive impairment a pharmaceutical composition according to the invention in an amount sufficient to alleviate or minimize the disease.

The potential application of sigma receptor ligands in the treatment of various CNS disease states and disorders such as substance abuse, Alzheimer's disease, stress, amnesia, depression, anxiety, psychosis, memory impairment is well documented. Subsequently, the claimed compounds may therefore also be used, but not limited to, the treatment of stress, depression, psychosis, memory impairment, schizophrenia and Alzheimer's disease. Additionally, the use of sigma ligands in treating various peripheral inflammatory conditions such as irritable bowel disease (IBD) has been claimed therefore the claimed compounds may therefore also be used, but not limited, to the treatment of various inflammatory conditions such as irritable bowel disease.

Accordingly, the invention also provides pharmaceutical compositions comprising the compounds of the invention and methods of treating and/or preventing the diseases set forth above.

In another embodiment the invention provides a pharmaceutical composition comprising a compound according to formula I

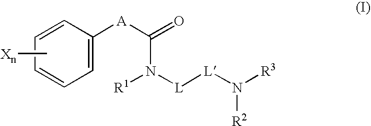

wherein,

X is halogen;

n is 0 to 5;

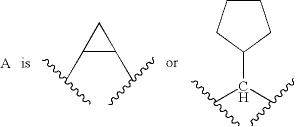

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

or a pharmaceutically acceptable salt thereof or steroisomeric forms thereof, and the geometrical isomers, enantiomers, diasteromers, and pharmaceutically acceptable salts thereof, together with a pharmaceutically acceptable diluent or carrier.

In another embodiment the invention provides a a pharmaceutical composition comprising a compound according to formula II

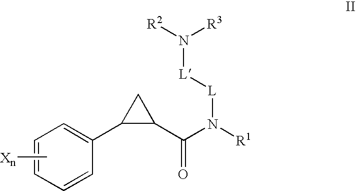

wherein,

X is halogen;

n is 0 to 5;

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

or a pharmaceutically acceptable salt thereof or steroisomeric forms thereof, and the geometrical isomers, enantiomers, diasteromers, and pharmaceutically acceptable salts thereof, together with a pharmaceutically acceptable diluent or carrier.

In another embodiment the invention provides a pharmaceutical composition comprising a compound according to formula III

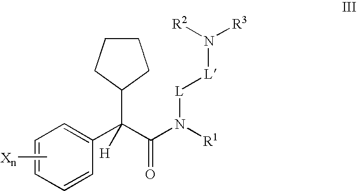

wherein,

X is halogen;

n is 0 to 5;

R¹ is hydrogen or can form together with L a heterocyclic ring;

R² is selected from hydrogen, C₁₋₈ alkyl straight or branched, C₃-C₈ cycloalkyl, or can form together with R³ a heterocyclic ring;

R³ is selected from hydrogen, C₁₋₈ alkyl straight or branched, C₃-C₈ cycloalkyl, or can form together with R² a heterocyclic ring;

L is selected from C₁₋₈ alkylene straight or branched, C₃-C₈ cycloalkyl, a direct bond or can from with R¹ a heterocyclic ring;

L′ is selected from C₁₋₈ alkylene straight or branched, C₃-C₈ cycloalkyl, a direct bond;

with the proviso that L and L′ are not both a direct bond;

or a pharmaceutically acceptable salt thereof or steroisomeric forms thereof, and the geometrical isomers, enantiomers, diasteromers, and pharmaceutically acceptable salts thereof, together with a pharmaceutically acceptable diluent or carrier.

Treatment or prevention can be carried out by administering to the patient an effective amount of one or more compounds according to the invention in a pharmaceutically acceptable carrier or diluent. The active materials can be administered by any appropriate route, for example, orally, parenterally, intravenously, intradermally, subcutaneously, intramuscularly or topically, in liquid, cream, gel or solid form, via a buccal or nasal spray, or aerosol.

Compounds of formula I and their salts can also be in the form of a solvate, which are also included within the scope of the present invention. Such solvates include for example hydrates, alcoholates and the like.

In accordance with the invention all stereoisomeric forms such as enantiomeric and diastereoisomeric forms of the compounds of formula I or mixtures thereof (including all possible mixtures of stereoisomers and racemates) are included. Typically one of the enantiomeric forms (eutomer) will be more therapeutically attractive than the other (distomer).

Unless otherwise stated the phrase “compounds according to the invention” as used herein refers collectively and individually to the compounds of formula I including geometrical isomers, enantiomers, diastereomers and racemates as well as pharmaceutically acceptable salts and complexes thereof, as well as preferred subsets thereof.

As used herein, the term pharmaceutically acceptable salts or complexes, refers to salts or complexes that retain the desired biological activity of the above-identified compounds and exhibit minimal undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as, but not limited to, acetic acid, oxalic acid, tartaric acid, succinic acid, fumaric acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The hydrochloric salt being the preferred salt. The compounds can also be administered as pharmaceutically acceptable quaternary salts known by those skilled in the art, which specifically include the quaternary ammonium salt of the formula —NR+Z-, wherein R is alkyl or benzyl, and Z is a counter ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Humans, equine, canine, bovine and other animals, and in particular, mammals, suffering from cough can be treated by administering to the patient an effective amount of one or more of the above-identified compounds or a pharmaceutically acceptable derivative or salt thereof in a pharmaceutically acceptable carrier or diluent to reduce formation of oxygen radicals. The active materials can be administered by any appropriate route, for example, orally, parenterally, transdermally, subcutaneously, buccal, intranasally, via aerosol or topically, in liquid, cream, gel or solid form. Preferably the compound is administered orally. Oral compositions will generally include an inert diluent or an edible carrier. They may be enclosed in gelatin capsules or compressed into tablets. For the purpose of oral therapeutic administration, the active compound can be incorporated with excipients and used in the form of tablets, lozenges, pills or capsules. Pharmaceutically compatible binding agents, and/or adjuvant materials can be included as part of the composition.

The tablets, pills or capsules can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

Solutions or suspensions used for parenteral, intradermal, subcutaneous, or topical application can include the following components: a sterile diluent such as saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

The active compound or pharmaceutically acceptable salt or derivative thereof can be administered as an aerosol preparation for inhalation which includes solutions and solids in powder form which may be in combination with a pharmaceutically acceptable carrier such as an inert compressed gas.

Liquid form preparations may also include solutions for intranasal and buccal administration.

Also included are solid form preparations which are intended to be converted, shortly before use, to liquid form preparations for either parenteral or oral administration. Such liquid forms include solutions, suspensions and emulsions.

The actual dosage employed may be varied depending on the requirements of the patient and the severity of the condition being treated. For the treatment of cough, a typical recommended dosage regimen is oral administration of from 10 mg-500 mg/day in two-four divided doses.

We have discovered that compounds claimed by this invention are potent novel Sigma-1 ligands which exhibit antitussive activity which make them useful for supressing cough in mammals. For mammals treated for cough, the sigma-1 receptor agonists may be administered along with one or more additional agents for treating cough, COPD, allergy or asthma symptoms selected from antihistamines, M3 antagonists, PDE4 inhibitors, 5-lipoxygenase inhibitors, leukotriene inhibitors, H3 receptor antagonists, β-adrenergic agonists, xanthine derivatives, α adrenergic agonists, mast cell stabilizers, antitussives, expectorants, VR-1 antagonists, Neurokinin receptor antagonists and GABA_{B} agonists. The sigma ligand and these additional agents are preferably administered in a combined dosage form (e.g single tablet) although they can be administered separately.

Non-limiting examples of antihistamines include astemizole, azatadine, cetirizine, clemastine, chlorpheniramine, hydroxyzine, mianserin, pyrilamine, terfenadine, loratidine and triprolidine.

Non-limiting examples of H3 receptor antagonists include impromidine, burimamide, mifetidine, and clozapine. Non-limiting examples of leukotriene inhibitors include monteleukast, pranlukast, zafirlukast. 5-Lipoxygenase inhibitors include any compound that inhibits restrains retards or otherwise interacts with the enzymatic action of 5-lipoxygenase. Non-limiting examples include zileuton and docebenone.

Non-limiting examples of β-adrenergic agonists include albuterol, perbuterol, terbutaline, isoproterenol and ephedrine.

A non-limiting example of a xanthine derivative is theophylline.

A non-limiting example of α-adrenergic agonist includes the arylalkylamines and imidazoles.

A non-limiting example of a mast cell stabilizer includes nedocromil sodium.

A non-limiting example of antitussive agents include codeine, dextromethorphan and noscapine.

A non-limiting example of an expectorant is guaifenesin.

A non-limiting example of Neurokinin receptor antagonist includes CP-99,994.

A non-limiting example of GABA_{B} agonist is baclofen.

As an “add on” treatment, the compounds of the invention can be administered together with drugs used to treat the CNS disorders (so for example together with acethylcholinesterase inhibitors for Alzheimers disease, together with serotonin and/or noradrenaline and/or dopamine reuptake inhibitors used to treat depression).

The present invention concerns also a method of treating or preventing conditions mediated by the sigma 1 receptor, the method comprising administering to a patient an amount of a compound having formula I or a pharmaceutically active derivative or salt thereof sufficient to prevent, reduce or eliminate the condition.

The present invention concerns also the use of a compound having general formula I, or a pharmaceutically active derivative or salt thereof for the manufacture of a medicament for a therapeutic application.

The following examples are provided for illustrative purposes only and are not intended, nor should they be construed as limiting the invention in any manner. Those skilled in the art will appreciate that variations and modifications of the following examples can be made without exceeding the spirit or scope of the invention.

### 2-cyclopentyl-2-phenyl-N-[3-(1-pyrrolidinyl)propyl]acetamide (compound 35)

Phenylcyclopentane carboxylic acid (500 mg, 2.45 mmol) was taken up in anhydrous dichloromethane (15 mL) and HBTU (1.0 g, 2.7 mmol) and N-methylmorpholine (0.3 mL, 2.7 mmol) were added to it and stirred under inert atmosphere for 5 min whereupon, 3-pyrrolidino propylamine (313 mg, 2.45 mmol) was added and the reaction was allowed to stir for 16 hours at room temperature. Upon completion of the reaction, the reaction mixture was washed with saturated sodium bicarbonate and DI water followed by drying over anhydrous magnesium sulfate. The solvent was removed in vacuo and the residue obtained was purified via flash column chromatography using chloroform/methanol as eluant. The free base obtained was then converted to hydrochloride salt via first dissolution in methanol followed by addition of trimethylsilyl chloride to afford the desired amide as hydrochloride salt (61 mg).

¹H NMR (CD₃OD, δ ppm): 0.9-1.2 (m, 1H), 1.2-1.35(m, 1H), 1.38-1.75(m, 5H), 1.8-2.2(m, 6H)2.5-2.7(m, 1H), 2.8-3.05(m, 4H), 3.15-3.26(m, 4H), 3.42-3.6(m, 2H), 7.15-7.35(m, 3H), 7.35-7.42(d, 2H).

### EXAMPLE 2

### N-{3-[4-(2-chloro-6-fluorobenzyl)-1-piperazinyl]propyl}-2-cyclopentyl-2-phenylacetamide (compound 34).

Trans-2-phenylcyclopropane carboxylic acid (5 g, 17.5 mmol) was dissolved in anhydrous tetrahydrofuran (25 mL) and carbonyldiimidazole (2.85 g, 17.5 mmol) was added to it. The reaction mixture was allowed to reflux for 1.5 h followed by addition of 3-[4-(2-chloro-6-fluorobenzyl)piperazino]propylamine (2.85 g, 17.5 mmol) and the reaction was allowed to stir at room temperature for 8 h. The solvent was removed in vacuo and redissolved in dichloromethane and washed with saturated sodium bicarbonate and water. The crude residue obtained after the removal of solvent was purified by passing through SCX-2 ion exchange column. Free base was converted to HCl salt by reacting with TMS-Cl in methanol to afford 7.45 g (85%) of the salt.

¹H NMR (CD₃OD, δ ppm): 1.22-1.27 (m, 1H), 1.45-1.50(m, 1H), 1.6-1.75(m, 2H), 1.75-1.82(m, 1H), 2.25-2.75(m, 1H), 3.15-3.25(m, 2H), 3.6-3.8(s, 2H), 7.1-7.4(m, 8H).

### EXAMPLE 3

### Sigma-1 Binding Assay

Membranes from guinea pig brain minus cerebellum were prepared according to that reported by Hellewell and Bowen (1990) Brain Res. 527 (2) 244-253. Briefly, 150-200 μg protein was incubated with [3H] (+) pentazocine (5 nM) (2R,6R,11R)- 1,2,3,4,5,6-hexahydro-6,11-dimethyl-3-(3-methyl-2-butenyl)-2,6-methano-3-benzazocin-8-ol, known sigma 1 radioligand, which can purchased from Alltech Product List and increasing concentrations of compounds to be tested in a buffer of the following composition 50 mM Tris-HCl, (pH 8.0). Samples were incubated for 2 hours at 37° C. Radiolabelled ligand bound to the membrane was harvested over 96 well GF/B microplates, purchased from Packard Bioscienc presoaked in 0.5% polyethyleneimine (PEI). Filters were washed 2 times with 5 mLs of ice-cold 10 μM Tris-HCl (pH 8.0). Non specific binding was determined in the presence of 10 μM (+) pentazocine. All assay points were performed in duplicate. Total binding was determined in the absence of competing cold ligand. Calculation of Kᵢ's and IC₅₀'s were made using well known methods. All of the claimed compounds have activity at the sigma-1 receptor of <10 uM. The more interesting compounds have affinity of less than 1 uM with the most interesting having affinity <400 nM.

### EXAMPLE 4

### In Vivo Cough Studies

The effects of the claimed Compounds(s) were evaluated in the citric-acid induced cough assay, a commonly used animal model to determine activity of potential antitussive drugs (Forsberg et al. Respiration l 992, 59 (2); 72-76).

Male Guinea pigs, weighing 400-450 grams were obtained from Elm Hill laboratories (Chelmsford, Mass., USA). Coughing was elicited in unanethestized guinea pigs by placement into individual plethysmographs and exposure to citric acid aerosol (400 mM). Dimensions of each chamber were approximately 9×27×9 cm. An ultrasonic nebulizer (Devilbiss) producing an aerosol with a mean particle size of 3.5 μM at a rate of approx. 0.2 ml/minute was used to deliver citric acid into the plethysmograph. The exposure period to the citric acid was 5 minutes followed by a further observation period of 9 minutes. The total number of coughs elicited by citric acid was determined over a total period of 14 minutes. Individual coughs were detected by two means: 1) via pressure transducer attached to the plethysmograph with amplification and recording onto a pen recorder and 2) via microphone detection. Since deflections often were not the result of a cough but rather a sneeze or deep breath, clarification of the cough response was additionally confirmed by the observer Analysis of each cough was achieved using appropriate software (Erika technologies, Paris, France). Since repetitive exposure of the animal did not elicit reproducible cough responses each animal was exposed to citric acid only once. Known sigma ligands (+)SKF 10,047 and BD-1047 were dissolved in saline (0.9%). (+) SKF 10,047 is a sigma 1 agonist (2S,6S, 11S)-(9Cl) 2,6-methano-3-benzazocin-8-ol, 1 ,2,3,4,5,6-hexahydro-6, 11-dimethyl-3-(2-propenyl), which can be purchased from Sigma_Aldrich or Wako BioProducts. BD 1047 is a sigma 1 antagonist N-[2-(3,4- dichlorophenyl)ethyl]-N-methyl-2-(dimethylamino)ethylaminoethylamine, which can be purchased ftom Tocris Cookson Product. Compounds of the present invention were dissolved in BEPP (1% ethanol, benzylalcohol, 2% benzylalcohol, 47 % Dextrose (5%)/F-168 (3.5%) mixture and 50% Peg 300/propylene glycol (80% /20%). Control animals were given the corresponding vehicle only. Individual guinea pigs were size matched and randomly allocated to each treatment group. Animals were then treated via intraperitoneal injection or oral gavage with either vehicle or test compound for 30 (i.p.) or 60 (p.o.) minutes immediately prior to citric acid aerosol exposure.

The compounds included in the present invention are claimed to inhibit cough by acting as sigma receptor agonists Consequently the selectivity of the claimed compounds to inhibit cough by this a sigma 1 agonist-mediated mechanism was confirmed by the administration of a selective antagonist, BD 1047 (5 mg/kg), of the sigma 1 receptor prior to administration of the claimed compound. The ability of this known antagonist to reverse the inhibition of cough was confirmed, in the presence of a known sigma-1 receptor agonist SKF 10,047.

### EXAMPLE 5

### Cognition Effect

The effect of claimed compounds has been evaluated in an animal model of short term/working memory, namely the spontaneous alternation task. The test system consisted of a symmetrical Y-shaped maze. Male mice have been used as experimental subjects and their behaviour observed in the maze during a period of 8 minutes In this situation, normal mice tended to spontaneously alternate the exploration of the 3 branches of the maze, i.e. they showed an ordered exploration sequence A-B-C or C-B-A (with arms arbitrarily designated A-B-C) in 65-70 % of the occasion (% spontaneous alternation). Mice treated with disocilpine (MK801), an NMDA antagonist that perturb memory processes, showed, as expected, a decreased percentage of spontaneous alternation indicating short term/working memory deficits. Claimed compounds were able to prevent this deficit after systemic administration.

Since deflections often were not the result of a cough but rather a sneeze or deep breath, clarification of the cough response was additionally confirmed by the observer. Analysis of each cough was achieved using appropriate software (Emka technologies, Paris, France). Since repetitive exposure of the animal did not elicit reproducible cough responses each animal was exposed to citric acid only once. Known sigma ligands (+)SKF 10,047 and BD-1047 were dissolved in saline (0.9%). (+) SKF 10,047 is a sigma 1 agonist (2S,6S,11S)-(9Cl) 2,6-methano-3-benzazocin-8-ol, 1,2,3,4,5,6-hexahydro-6,11 -dimethyl-3-(2-propenyl), which can be purchased from Sigma_Aldrich or Wako BioProducts. BD 1047 is a sigma 1 antagonist N-[2-(3,4-dichlorophenyl)ethyl]-N-methyl-2-(dimethylamino)ethylamino)ethylamine, which can be purchased from Tocris Cookson Product. Compounds of the present invention were dissolved in BEPP (1% ethanol, benzylalcohol, 2% benzylalcohol, 47% Dextrose (5%)/F-168 (3.5%) mixture and 50% Peg 300/propylene glycol (80% /20%). Control animals were given the corresponding vehicle only. Individual guinea pigs were size matched and randomly allocated to each treatment group. Animals were then treated via intraperitoneal injection or oral gavage with either vehicle or test compound for 30 (i.p.) or 60 (p.o.) minutes immediately prior to citric acid aerosol exposure.

The compounds included in the present invention are claimed to inhibit cough by acting as sigma receptor agonists. Consequently the selectivity of the claimed compounds to inhibit cough by this a sigma 1 agonist-mediated mechanism was confirmed by the administration of a selective antagonist, BD 1047 (5 mg/kg), of the sigma 1 receptor prior to administration of the claimed compound. The ability of this known antagonist to reverse the inhibition of cough was confirmed, in the presence of a known sigma-1 receptor agonist SKF 10,047.

### EXAMPLE 5

### Cognition Effect

The effect of claimed compounds has been evaluated in an animal model of short term/working memory, namely the spontaneous alternation task. The test system consisted of a symmetrical Y-shaped maze. Male mice have been used as experimental subjects and their behaviour observed in the maze during a period of 8 minutes. In this situation, normal mice tended to spontaneously alternate the exploration of the 3 branches of the maze, i.e. they showed an ordered exploration sequence A-B-C or C-B-A (with arms arbitrarily designated A-B-C) in 65-70 % of the occasion (% spontaneous alternation). Mice treated with disocilpine (MK801), an NMDA antagonist that perturb memory processes, showed, as expected, a decreased percentage of spontaneous alternation indicating short term/working memory deficits. Claimed compounds were able to prevent this deficit after systemic administration.

1. A pharmaceutical composition comprising a compound of formula I 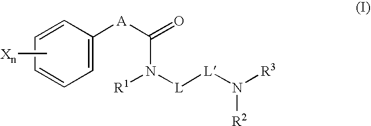
wherein,
X is halogen;
n is O to 5; 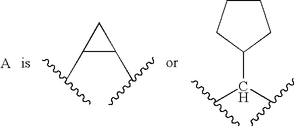
Rhu 1 is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
or a pharmaceutically acceptable salt thereof or stereoisomeric forms thereof, and the geometrical isomers, enantiomers, diastereoisomersor their corresponding N-oxides and pharmaceutically acceptable salts thereof with a pharmaceutically acceptable diluent or carrier.

2. A pharmaceutical composition according to claim 1, comprising a compound of formula II 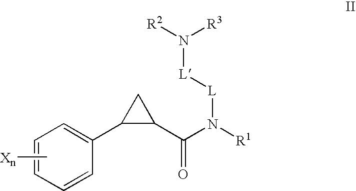
wherein,
X is halogen;
n is 0 to 5;
R¹ is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
or a pharmaceutically acceptable salt thereof or stereoisomeric forms thereof, and the geometrical isomers, enantiomers, diastereoisomers, and pharmaceutically acceptable salts thereof with a pharmaceutically acceptable diluent or carrier.

3. A pharmaceutical composition according to claim 1, comprising a compound of formula III 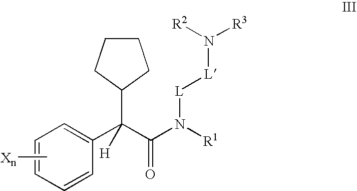
wherein,
X is halogen;
n is 0 to 5;
R¹ is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
or a pharmaceutically acceptable salt thereof or stereoisomeric forms thereof, and the geometrical isomers, enantiomers, diastereoisomers, and pharmaceutically acceptable salts thereof with a pharmaceutically acceptable diluent or carrier.

4. A composition according to claim 1 wherein R² and R³ are ethyl, piperidyl, pyrrolidyl, hydrogen, cyclohexyl.

5. A pharmaceutical composition according to claim 1, comprising a hydrocloride salt of a compound selected from 2-cyclopentyl-N-[3-(2-methyl-1-piperidinyl)propyl]-2-phenylacetamide; 2-cyclopentyl-N-[2-(diethylamino)ethyl]-2-phenylacetamide; (−) 2-cyclopentyl-N-[2-(diethylamino)ethyl]-2-phenylacetamide; (+) 2-cyclopentyl-N-[2-(diethylamino)ethyl]-2-phenylacetamide; trans-N-[2-(diisopropylamino)ethyl]-2-phenylcyclopropanecarboxamide; 2-cyclopentyl-N-[2-(diethylamino)ethyl]-2-(4-fluorophenyl)acetamide; 2-cyclopentyl-N-[3-(diethylamino)propyl]-2-phenylacetamide; 1′-[cyclopentyl(phenyl)acetyl]-1,4′-bipiperidine; 1′-[cyclopentyl(3,4-dichlorophenyl)acetyl]-1,4′-bipiperidine; 1-[cyclopentyl(phenyl)acetyl]-4-pyrrolidin-1-ylpiperidine; 1′-[(4-chlorophenyl)(cyclopentyl)acetyl]-1,4′-bipiperidine; 2-cyclopentyl-2-phenyl-N-[2-(1-pyrrolidinyl)ethyl]acetamide; trans-N-{3-[4-(2-chloro-6-fluorobenzyl)-1-piperazinyl]propyl}-2-cyclopentyl-2-phenylacetamide; trans-N-[3-(cyclohexylamino)propyl]-2-phenylcyclopropanecarboxamide; trans-1′-[(2-phenylcyclopropyl)carbonyl]-1,4′-bipiperidine; trans-2-phenyl-N-(2-pyrrolidin-1-ylethyl)cyclopropanecarboxamide; 2-cyclopentyl-2-phenyl-N-[3-(1-pyrrolidinyl)propyl]acetamide; trans-N-{3-[4-(2-chloro-6-fluorobenzyl)-1-piperazinyl]propyl}-2-phenylcyclopropanecarboxamide; trans-N-[2-(4-benzyl-1-piperazinyl)ethyl]-2-phenylcyclopropanecarboxamide; trans-N-(cyclohexylmethyl){1-[(2-phenylcyclopropyl)carbonyl]-4-piperidinyl}methanamine; trans-N-{2-[4-(3,4-dichlorobenzyl)-1- piperazinyl]ethyl}-2-phenylcyclopropanecarboxamide; trans-1′-[(2-phenylcyclopropyl)carbonyl]-1,4′-bipiperidine; trans-N-[2-(diethylamino)ethyl]-2-phenylcyclopropanecarboxamide; N-[3-(cyclohexylamino)propyl]-2-cyclopentyl-2-phenylacetamide; trans-(+)N-[3-(cyclohexylamino)propyl]-2-phenylcyclopropanecarboxamide; trans-(−)N-[3-(cyclohexylamino)propyl]-2-phenylcyclopropane carboxamide.

6. A pharmaceutical composition according to claim 1, comprising a hydrochloride salt of a compound selected from 2-cyclopentyl-N-[2-(diethylamino)ethyl]-2-phenylacetamide; (−) 2-cyclopentyl-N-[2-(diethylamino)ethyl]-2-phenylacetamide; (+) 2-cyclopentyl-N-[2-(diethylamino)ethyl]-2-phenylacetamide; 2-cyclopentyl-N-[3-(diethylamino)propyl]-2-phenylacetamide; 1′-[cyclopentyl(phenyl)acetyl]-1,4′-bipiperidine; 1′-[cyclopentyl(3,4-dichlorophenyl)acetyl]-1,4′-bipiperidine; 1-[cyclopentyl(phenyl)acetyl]-4-pyrrolidin-1-ylpiperidine; trans-N-[3-(cyclohexylamino)propyl]-2-phenylcyclopropanecarboxamide; trans-1′-[(2-phenylcyclopropyl)carbonyl]-1,4′-bipiperidine; trans-(+)N-[3-(cyclohexylamino)propyl]-2-phenylcyclopropanecarboxamide; trans-(−)N-[3-(cyclohexylamino)propyl]-2-phenylcyclopropanecarboxamide.

7. A pharmaceutical composition according to claim 1, comprising a hydrochloride salt of a compound selected from trans-N-[3-(cyclohexylamino)propyl]-2-phenylcyclopropanecarboxamide; trans-(+)N-[3-(cyclohexylamino)propyl]-2-phenylcyclopropanecarboxamide; trans-(−)N-[3-(cyclohexylamino)propyl]-2-phenylcyclopropanecarboxamid;. 2-cyclopentyl-N-[2-(diethylamino)ethyl]-2-phenylacetamide; (−) 2-cyclopentyl-N-[2-(diethylamino)ethyl]-2-phenylacetamide; (+) 2-cyclopentyl-N-[2-(diethylamino)ethyl]-2-phenylacetamide.

8. A compound having formula I or a pharmaceutically acceptable salt thereof or steroisomeric forms thereof, and the geometrical isomers, enantiomers, diastereoisomers, or their corresponding N-oxides and pharmaceutically acceptable salts thereof 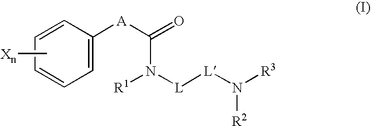
wherein,
X is halogen;
n is 0 to 5; 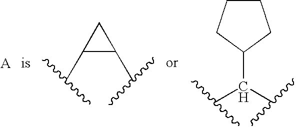
R¹ is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
except compounds N-3-[-(dimethylamino) propyl]-2-phenyl-cyclopropane carboxamide; N-[2-(4-morpholinyl)ethyl]-2-phenyl-cyclopropanecarboxamide; N-2-[-(dimethyl amino) ethyl]-2-phenyl-cyclopropanecarboxamide; N-[3-(4-morpholinyl) propyl]-2- phenyl-cyclopropane carboxamide; 2-phenyl-N-[2-(1-piperidinyl)ethyl]-cyclopropanecarboxamide; (1R,2R)-N-(4-aminobutyl)-2-phenyl-cyclopropanecarboxamide; 1-[[(1R,2R)-2-phenyl cyclopropyl] carbonyl]-4- piperidine methanamine mono (trifluoroacetate); N-(4-aminobutyl)-2-phenyl-cyclopropanecarboxamide; 2-cyclopentyl-N-[3-(2,6-dimethyl-piperidin-1-yl)-propyl]-2-phenyl-acetamide; 2-cyclopentyl-N-(1-diethylaminomethyl-cyclohexyl)-2-phenyl-acetamide.

9. A compound according to claim 8 having formula II or a pharmaceutically acceptable salt thereof or steroisomeric forms thereof, and the geometrical isomers, enantiomers, diastereoisomers, and pharmaceutically acceptable salts thereof 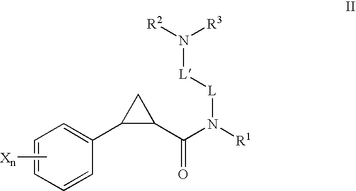
wherein,
X is halogen;
n is 0 to 5;
R¹ is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
except N-3-[-(dimethylamino)propyl]-2-phenyl-cyclopropanecarboxamide; N-[2-(4-morpholinyl)ethyl]-2-phenyl-cyclopropanecarboxamide; N-2-[-(dimethyl amino) ethyl]-2-phenyl-cyclopropanecarboxamide; N-[3-(4-morpholinyl)propyl]-2-phenyl-cyclopropanecarboxamide; 2-phenyl-N-[2-(1-piperidinyl)ethyl]-cyclopropanecarboxamide; (1R,2R)-N-(4-aminobutyl)-2-phenyl-cyclopropanecarboxamide; 1-[[(1R,2R)-2-phenyl cyclopropyl]carbonyl]-4-piperidine methanamine mono-trifluoroacetate; N-(4-aminobutyl)-2-phenyl-cyclopropanecarboxamide;

10. A compound according to claim 8 having formula III or a pharmaceutically acceptable salt thereof or steroisomeric forms thereof, and the geometrical isomers, enantiomers, diastereoisomers, and pharmaceutically acceptable salts thereof 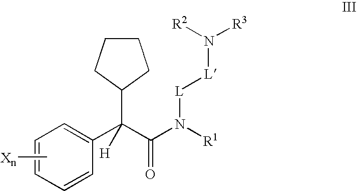
wherein,
X is halogen;
n is 0 to 5;
R¹ is hydrogen or can form together with L a heterocyclic ring;
R² is selected from hydrogen, C₁₋₈ alkyl straight or branched, C₃-C₈ cycloalkyl, or can form together with R³ a heterocyclic ring;
R³ is selected from hydrogen, C₁₋₈ alkyl straight or branched, C₃-C₈ cycloalkyl, or can form together with R² a heterocyclic ring;
L is selected from C₁₋₈ alkylene straight or branched, C₃-C₈ cycloalkyl, a direct bond or can from with R¹ a heterocyclic ring;
L′ is selected from C₁₋₈ alkylene straight or branched, C₃-C₈ cycloalkyl, a direct bond;
with the proviso that L and L′ are not both a direct bond;
except 2-cyclopentyl-N-[3-(2,6-dimethyl-piperidin-1-yl)propyl]-2-phenyl-acetamide; 2-2yclopentyl-N-(1-diethylaminomethyl-cyclohexyl)-2-phenylacetamide.

11. A compound according to claim 8 wherein R² and R³ are ethyl, piperidyl, pyrrolidyl, hydrogen, cyclohexyl.

12. A hydrochloride salt of a compound according to claim 8 selected from: 2-cyclopentyl-N-[3-(2-methyl-1-piperidinyl)propyl]-2-phenylacetamide; 2-cyclopentyl-N-[2-(diethylamino)ethyl]-2-phenylacetamide; (−) 2-cyclopentyl-N-[2-(diethylamino)ethyl]-2-phenylacetamide; (+) 2-cyclopentyl-N-[2-(diethylamino)ethyl]-2-phenylacetamide; trans-N-[2-(diisopropylamino)ethyl]-2-phenylcyclopropanecarboxamide; 2-cyclopentyl-N-[2-(diethylamino)ethyl]-2-(4-fluorophenyl)acetamide; 2-cyclopentyl-N-[3-(diethylamino)propyl]-2-phenylacetamide; 1′-[cyclopentyl(phenyl)acetyl]-1,4′-bipiperidine; 1′-[cyclopentyl(3,4-dichlorophenyl)acetyl]-1,4′-bipiperidine; 1-[cyclopentyl(phenyl)acetyl]-4-pyrrolidin-1-yipiperidine; 1′-[(4-chlorophenyl)(cyclopentyl)acetyl]-1,4′-bipiperidine; 2-cyclopentyl-2-phenyl-N-[2-(1-pyrrolidinyl)ethyl]acetamide; trans-N-{3-[4-(2-chloro-6-fluorobenzyl)-1-piperazinyl]propyl}-2-cyclopentyl-2-phenylacetamide; trans-N-[3-(cyclohexylamino)propyl]-2-phenylcyclopropanecarboxamide; trans-1′-[(2-phenylcyclopropyl)carbonyl]-1,4′-bipiperidine; trans-2-phenyl-N-(2-pyrrolidin-1-ylethyl)cyclopropanecarboxamide (free base); 2-cyclopentyl-2-phenyl-N-[3-(1-pyrrolidinyl)propyl]acetamide; trans-N-{3-[4-(2-chloro-6-fluorobenzyl)-1-piperazinyl]propyl}-2-phenylcyclopropanecarboxamide; trans-N-[2-(4-benzyl-1-piperazinyl)ethyl]-2-phenylcyclopropanecarboxamide; trans-N-(cyclohexylmethyl){1-[(2-phenylcyclopropyl)carbonyl]-4-piperidinyl}methanamine; trans-N-{2-[4-(3,4-dichlorobenzyl)-1-piperazinyl]ethyl}-2-phenylcyclopropanecarboxamide; trans-1′-[(2-phenylcyclopropyl)carbonyl]-1,4′-bipiperidine; trans-N-[2-(diethylamino)ethyl]-2enylcyclopropanecarboxamide; N-[3-(cyclohexylamino)propyl]-2-cyclopentyl-2-phenylacetamide; trans-(+)N-[3-(cyclohexylamino)propyl]-2-phenylcyclopropanecarboxamide; trans-(−)N-[3-(cyclohexylamino)propyl]-2-phenylcyclopropanecarboxamide.

13. A hydrochloride salt of a compound according to claim 8 selected from 2-cyclopentyl-N-[2-(diethylamino)ethyl]-2-phenylacetamide; (−) 2-cyclopentyl-N-[2-(diethylamino)ethyl]-2-phenylacetamide; (+) 2-cyclopentyl-N-[2-(diethylamino)ethyl]-2-phenylacetamide; 2-cyclopentyl-N-[3-(diethylamino)propyl]-2-phenylacetamide; 1′-[cyclopentyl(phenyl)acetyl]-1,4′-bipiperidine; 1′-[cyclopentyl(3,4-dichlorophenyl)acetyl]-1,4′-bipiperidine 1-[cyclopentyl(phenyl)acetyl]-4-pyrrolidin-1-ylpiperidine; trans-N-[3-(cyclohexylamino)propyl]-2-phenylcyclopropanecarboxamide; trans-1′-[(2-phenylcyclopropyl)carbonyl]-1,4′-bipiperidine; trans-(+)N-[3-(cyclohexylamino)propyl]-2-phenylcyclopropanecarboxamide; trans-(−)N-[3-(cyclohexylamino)propyl]-2-phenylcyclopropanecarboxamide.

14. A hydrochloride salt of a compound according to claim 8 selected from trans-N-[3-(cyclohexylamino)propyl]-2-phenylcyclopropanecarboxamide; trans-(+)N-[3-(cyclohexylamino)propyl]-2-phenylcyclopropanecarboxamide trans-(−)N-[3-(cyclohexylamino)propyl]-2-phenylcyclopropanecarboxamid (−) 2-cyclopentyl-N-[2-(diethylamino)ethyl]-2-phenylacetamide; (+) 2-cyclopentyl-N-[2-(diethylamino)ethyl]-2-phenylacetamide.

15. A method for treating or preventing conditions mediated by the sigma 1 receptor, the method comprising administering to a patient an amount of a compound of formula I as defined in any of claim 1 sufficient to prevent, reduce or eliminate the condition.

16. A method according to claim 15 wherein the condition is selected from cough, Alzheimers, depression, psychosis, stress, senescence and memory impairment, immune modulation, inflammation, and diseases of cognitive dysfunction.

17. A method according to claim 15 wherein the condition is cough.

18. A method according to claim 15 wherein the condition is memory impairment.

19. A method according to claim 15 wherein the condition is disease of cognitive dysfunction.
